(19) United States
(12) Patent Application Publication
(10) Pub. No.: US 2011/0297456 A1
(43) Pub. Date: Dec. 8, 2011

(54) Scales Displaying Nutritional Information

(75) Inventors: Lucy Davidson, Berkshire (GB); Corrina Griffin, Berkshire (GB)

(73) Assignee: Weight Watchers International, Inc., Woodbury, NY (US)

(21) Appl. No.: 13/210,445

(22) Filed: Aug. 16, 2011

Related U.S. Application Data
(63) Continuation of application No. 11/896,594, filed on Sep. 4, 2007, now abandoned.
Application No. 11/896,594 is a continuation of application No. 10/431,562, filed on May 8, 2003, now abandoned.

(30) Foreign Application Priority Data
Dec. 18, 2002 (GB) 0229467.6

(51) Int. Cl. G01G 19/40; G01N 33/02

(52) U.S. Cl. 177/1; 177/25.16

(57) ABSTRACT

A scale provides a user with a display of a nutritional parameter on the basis of the weight of the food being weighed. The values for a plurality of food items can be accumulated and a single nutritional parameter characterizing a complete meal or recipe may be displayed.

This application claims priority under 35 U.S.C. §§119 and/or 365 to United Kingdom Patent Application No. 0229467.6 filed in Great Britain on Dec. 18, 2002; the entire content of which is hereby incorporated by reference.

### FIELD OF THE INVENTION

The present invention relates to scales which display nutritional information.

### BACKGROUND TO THE INVENTION

British Patent GB-A-2133166 discloses an electronic scale which is provided with means to enable a user to identify food being weighed. This information is then used, together with a weight value, to generate a value for the caloric value of the food being weighed. However, the scale proposed in GB-A-2133166 is only capable of providing a caloric value for one food item at a time. In contrast, most meals and recipes consist of a combination of ingredients, for example, a meal of roast chicken, peas and boiled potatoes or a cake recipe.

U.S. Pat. Nos. 6,040,531 and 6,436,036 disclose devices and methods for calculating and tallying single-valued nutritional indicators for known quantities of various foodstuffs. The single-valued nutritional indicator (such as those expressed in terms of POINTS®) may take into account multiple nutritional properties such as caloric content, fat content and fiber content. However, the devices of these patents have no mechanism for determining the single-valued nutritional indicators for unknown quantities of a foodstuff or combinations of foodstuffs such as might, for example, be spooned out on a serving plate for an evening meal.

### SUMMARY OF THE PREFERRED EMBODIMENTS

It is an aim of the present invention to provide a scale which is of enhanced utility to those for whom the nutritional parameters of their food are important. Preferred embodiments of the present invention provide single-valued nutritional indicators for a weighed foodstuff or combinations of foodstuffs, the total single-valued nutritional indicator taking into account multiple nutritional attributes of the foodstuffs.

According to the present invention, there is provided a scale comprising

a weight sensor;

a database memory storing a database of specific nutritional parameter values for a plurality of foodstuffs;

a display system such as a keypad or touch screen;

user input system; and

a processor configured to be responsive to operations of the user input system to select data from the memory and generate respective absolute nutritional parameter values or single-valued nutritional indicators in dependence on the output of the weight sensor and the data. In preferred embodiments, a single-valued nutritional indicator per unit weight is stored in a look-up table in the database memory for various common food stuffs.

In preferred embodiments the processor is configured for accumulating a plurality of single-valued nutritional indicators, generated in response to a plurality of user input operations using the user input system, and controlling the display to display the resultant total accumulated single-valued nutritional indicator for foods assembled on the scale.

The terms “specific” and “absolute” as used herein mean “per unit” and “total”.

The weight sensor could be of any type that gives an output received by the processor, for example an electronic load cell.

The term “database” is used only to indicate a source of organized data and may be as simple as a lookup table or list of per unit weight values. Alternatively, the database may contain underlying nutritional data such as caloric content per unit weight, fat per unit weight etc. from which a single-valued nutritional indicator can be calculated.

The processor preferably comprises a microprocessor or a microcontroller. However, an ASIC could be employed.

The user input system preferably comprises a keypad. However, it could comprise a touch sensitive display or a display in association with a pointing device, such as a mouse or tracker ball.

Preferably, the processor is configured to be responsive to a user input operation, using the user input system, to store a generated absolute nutritional parameter value in a writable memory, e.g. RAM, and to include absolute nutritional parameter values stored in the writable memory in said accumulated nutritional parameter value. The writable memory may comprises one or more discrete devices or be integrated into a processor, such as a microcontroller.

Preferably, the processor means is configured for storing a foodstuff identifier in the writable memory in association with each generated absolute nutritional parameter value stored therein. More preferably, the processing means is configured to be responsive to a user input operation, using the user input means, to read a foodstuff identifier and an absolute nutritional parameter value from the writable memory and control the display to display an indication of the foodstuff identified by said identifier and the associated absolute nutritional parameter value. The foodstuff identifier stored in the writable memory need not be the actual name of the foodstuff and the indication displayed could be selected from a list of foodstuff names on the basis of the identifier. Still more preferably, the processor is configured to be responsive to a user input operation, using the user input system, while said indication and associated nutritional parameter are being displayed, to update the corresponding nutritional parameter value in the writable memory in dependence on the output of the weight sensor.

Preferably, the processor means is configured to be responsive to a user input operation, using the user input system, to read a foodstuff identifier from database memory and control the display to display the name of the foodstuff identified by said identifier. More preferably, the processing means is configured to be responsive to user input operations, using the user input means, to read foodstuff identifiers sequentially from database memory and control the display to display in sequence the name of each foodstuff identified by one of said identifiers. Thus, the user can scroll through the stored items. Still more preferably, the user input system includes character input means and the processor means is configured to be responsive to user input operations, using character user means, of a sequence of characters to select read a foodstuff identifier from the database memory for which the start of the name of the foodstuff corresponds to the input character sequence.

The nutritional parameter or single-valued nutritional indicator for a foodstuff may be defined by algorithms such as disclosed in U.S. Pat. Nos. 6,040,531 and 6,436,036 to Miller-Kovach et al., the contents of which are hereby incorporated by reference herein in their entirety. In one embodiment single-valued nutritional indicators may be determined and tallied in terms of “POINTS®.” POINTS® is a registered trademark of Weight Watchers International. One such algorithm is:

$$p = \frac{c}{{k}_{1}} + \frac{f}{{k}_{2}}$$

where c is the calorific value of the foodstuff in kilocalories per gram, f is the total fat content of the food stuff per gram in grams, k₁ is constant, preferably about 70, k₂ is in the range k₁/35 to k₁/10, preferably k₁/17.5.

Alternatively, the specific nutritional parameter for a foodstuff may be defined by:

$$p = \frac{c}{{k}_{1}} + \frac{f}{{k}_{2}} - \frac{r}{{k}_{3}}$$

where c is the calorific value of the foodstuff in kilocalories per gram, f is the total fat content of the food stuff per gram in grams, r is the dietary fiber content of the foodstuff in grams per gram, k₁ is constant, preferably about 50, k₂ is in the range k₁/3 to k₁/5, preferably k₁/4, and k₃ is in the range k₁/20 to k₁/5, preferably k₁/10.

The present invention also relates to methods for providing a total, single-valued nutritional indicator for a food serving or recipe containing plural foodstuffs using a scale with a weight sensor, database user input, processor and display. In accordance with such methods the following steps may be performed:

(a) placing a food vessel on the scale;

(b) zeroing the weight reading of the weight sensor;

(c) placing a foodstuff in the vessel;

(d) entering an identification of the foodstuff using the user input;

(e) employing the processor and database to provide a selected nutritional indicator per unit weight of the identified foodstuff and calculating a single-valued nutritional indicator for the weight of the identified foodstuff sensed by the weight sensor;

(f) repeating steps (b)-(e) at least once;

(g) calculating a total single-valued nutritional indicator for the foodstuffs in the vessel;

(h) displaying the total, single-valued nutritional indicator for the foodstuffs in the vessel.

The method may also include the step of displaying a current total, single-valued nutritional indicator for the foodstuffs in the vessel after each foodstuff is added to the vessel. The indicator may advantageously may be displayed to an accuracy of one decimal place or more.

The foregoing is intended as a convenient summary of preferred embodiments of the present invention. The scope of the invention is, however, determined by the claims.

### BRIEF DESCRIPTION OF THE DRAWINGS

FIGS. 1(a) and 1(b) are perspective views of two scale embodiments according to the present invention;

FIG. 2 is a block diagram of an embodiment of the electronics of the scales of FIG. 1; and

FIG. 3 is a state diagram illustrating the operation of the scales of FIG. 1.

### DETAILED DESCRIPTION OF PREFERRED EMBODIMENTS

Embodiments of the present invention will now be described, by way of example, with reference to the accompanying drawings.

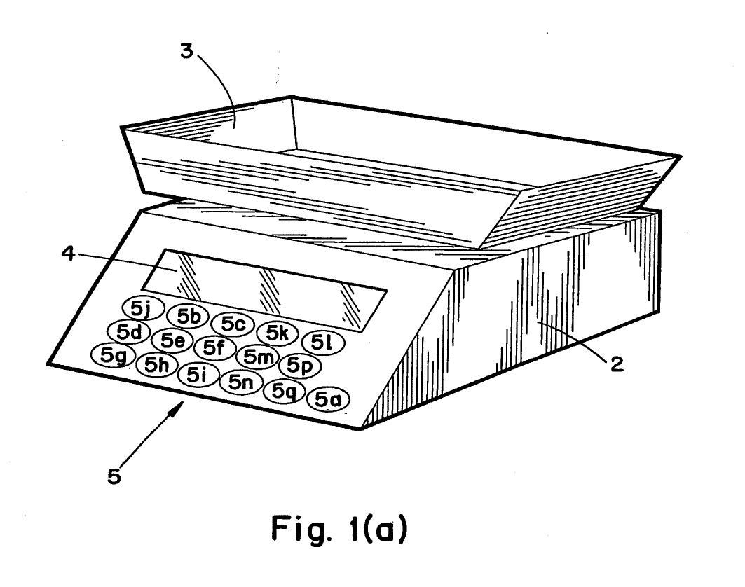

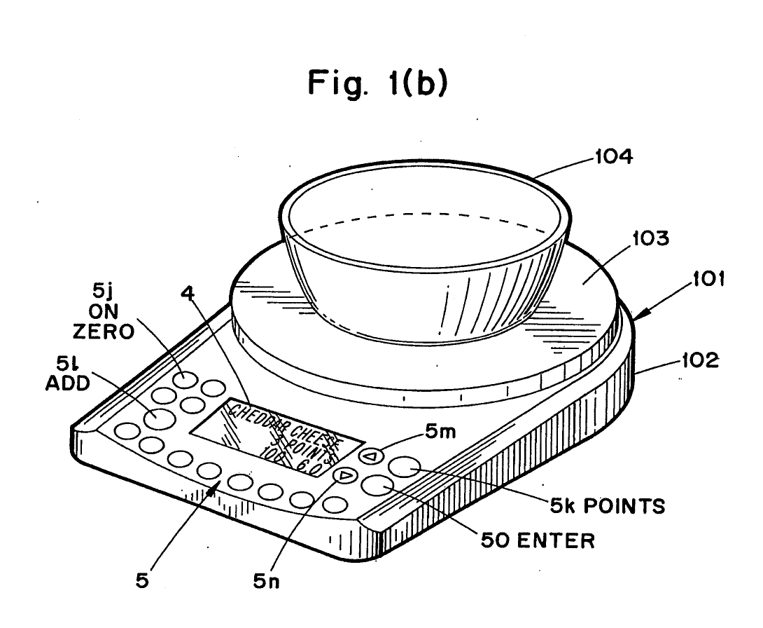

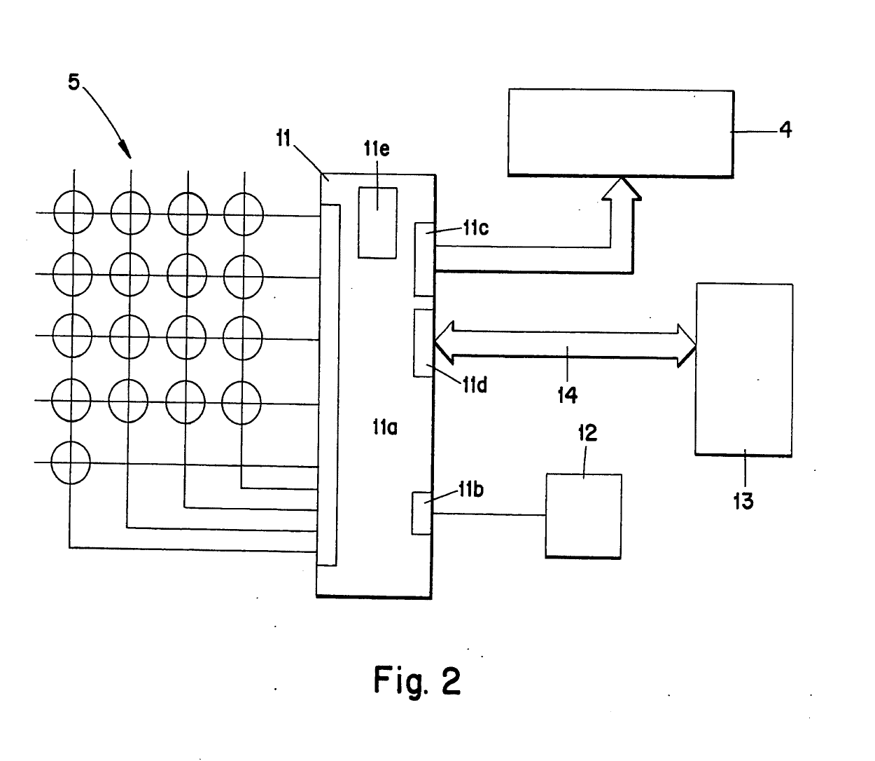

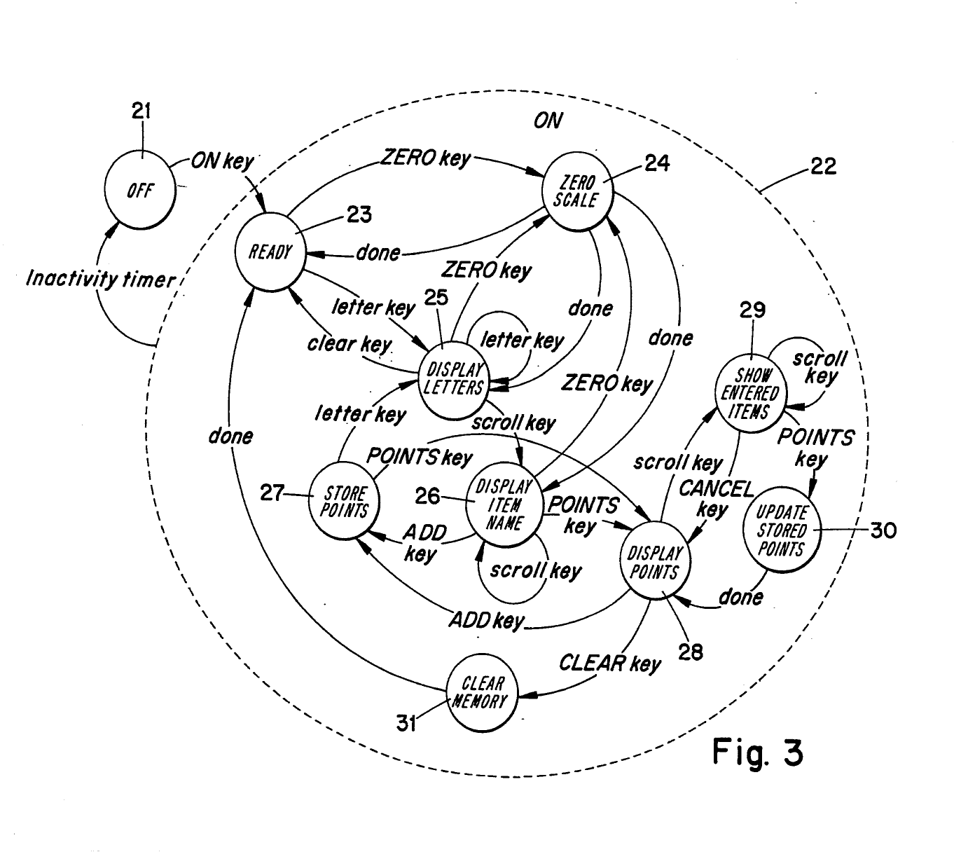

Referring to FIG. 1(a), a scale 1 comprises a body 2 and a pan 3 supported on a load cell 12 (FIG. 2) within the body 2 by a coupling member passing through the top of the body. The body 2 has a sloping front face which contains an LCD (liquid crystal display) 4 and a keypad 5. In addition to the load cell, the body 2 houses microcomputer circuitry.

Referring to FIG. 1(b), a scale 101 comprises a body 102 and a platen 103 supported on a load cell 12 (FIG. 2) within the body 102 by a coupling member passing through the top of the body. The body 102 has a face which carries an LCD (liquid crystal display) 4 and a keypad 5. In addition to the load cell, the body 102 houses microcomputer circuitry.

The platen 103 is adapted for receiving a food vessel 104 such as a dinner plate or a mixing bowl as shown in FIG. 1(b). In operation the vessel 104 may be placed on the platen 103 and the scale zeroed by pressing the “ON/ZERO” button on the keypad. The display then registers a weight of “0” grams or ounces. A recipe ingredient such as cheese may then be added to the vessel and only its weight will be registered. The user may then identify the foodstuff using alphanumeric keys on the keypad. The processor selects a per unit weight value appropriate for the identified foodstuff and calculates a single-valued nutritional indicator such as the POINTS® value for the weight of the foodstuff placed in the vessel. As shown in FIG. 1(b), for example, the foodstuff is identified as “CHEDDAR CHEESE,” its weight is displayed as “100 grams” and its POINTS® value displayed as “6.0.” The scale may be again zeroed and a second foodstuff placed in the vessel with the cheese. The foodstuff may be identified by the user and a total POINTS® value for the cheese and the second foodstuff calculated and displayed. In this manner the scale may be operated in an “add and weigh” mode to add a particular weight or point value of each ingredient, and to assemble all of the ingredients in the vessel and display a total POINTS® value for the assembled ingredients.

In a similar fashion, a dinner plate may be used as the vessel, and a dinner serving of various foodstuffs assembled while keeping a running total of the POINTS® value for the various foods being assembled on the plate.

Referring to FIG. 2, the circuitry of the scale is based around a microcontroller 11 which includes a keypad interface 11a, an analog-to-digital converter 11b, an LCD interface 11c, a data/address bus interface 11d and internal RAM 11e. The keypad interface 11a is connected to the keys 5 and the LCD interface 11c is connected to the LCD 4. The output of the load cell 12 is connected to the analog-to-digital converter 11b. A ROM 13 is connected to the data/address bus 11d by a data/address bus 14.

The ROM is programmed with a database of food types and associated specific, i.e. per unit weight, nutritional parameter values. In the present embodiment, the specific nutritional parameter values may be POINTS® per gram where a POINTS® value p for a food item is defined by:

$$p = \frac{c}{{k}_{1}} + \frac{f}{{k}_{2}}$$

where c is the calorific value of the food item in kilocalories, f is the saturated fat content in grams, k₁ is a 70 and k₂ is 4.

The keypad 5 may comprise an ON key 5a, an ABC key 5b, a DEF key 5c, a GHI key 5d, an JKL key 5e, an MNO key 5f, a PQRS key 5g, a TUV key 5h, a WXYZ key 5i, a ZERO key 5j, a POINTS key 5k, an ADD key 5l, an UP scroll key 5m, a DOWN scroll key 5n, an ENTER key 5o, a CLEAR key 5p and a CANCEL key 5q.

The microcontroller 11 is programmed to determine weights and POINTS® values for food items on the basis of the output of the load cell 12 and user inputs made using the keypad 5 and to accumulate POINTS® values. The skilled person will discern the nature of this programming from the following description of the operational states of the scale 1.

Referring to FIG. 1, the scale 1 is activated by the user pressing the ON key 5a. This moves the scale 1 from an off state 21 to an on state 22. The on state 22 comprises a plurality of substrates 23, . . . , 31. A timer implemented by the microcontroller 11 generates a timeout interrupt if the scale 1 is in the on state 22 and no key presses have occurred for 3 minutes. The microcontroller 11 responds to this timeout interrupt by returning the scale 1 to the off state 21.

When the scale is in the on state 22, the microcontroller 11 regularly reads the output of its analog-to-digital converter 11b, converts the output of the into a grams (or ounces) value and drives the LCD 4 to display, the grams value. Thus, with nothing placed in the pan 2, the LCD displays 0 g. However, if an object is placed in the pan 2, the output of the load cell 12 will change causing the output of the analogue-to-digital converter 11b to change and a new non-zero weight will be displayed by the LCD 4.

The pressing of the keys of the keypad 5 triggers interrupt handlers and the response of the microcontroller 11 to any key press will be determined by the identity of the key pressed and the state that the microcontroller 11 is currently in. The ABC, DEF etc keys 5b, . . . , 5i operate in a manner familiar from mobile phones. If a user wishes to input a B, the user presses the ABC key 5b twice in rapid succession. Similarly, if the user wants to input a T, the user presses the TUV key 5h once. The operation of any of the ABC, DEF etc keys 5b, . . . , 5i triggers a timer in the microcontroller 11 and the microcontroller does not settle on the input letter until the timer has expired without one of the ABC, DEF etc keys 5b, . . . , 5i being pressed. In the interests of clarity, the input of letters using multiple key presses will be treated as a single event in the following description of the operation of the scale 1.

On entering the on state 22, the scale is placed in a ready state 23 in which the current weight is displayed on the LCD 4.

If a letter key 5b, . . . , 5i is pressed in the ready state 23, the scale 1 moves to a display letters state 24 in which the input letter is displayed. Further operations of letter keys 5b, . . . , 5k in the display letters state 25 add letters to the display. Pressing the CLEAR key 5p in the display letters state 25 deletes the most recently input letter and, if the last letter is removed, returns the scale 1 to the ready state 23.

If one of the scroll keys 5m, 5n is pressed in the display letters state 25, the microcontroller 11 will attempt to move the scale 1 into a display item name state 26. The microcontroller 11 responds to the scroll key press by finding respectively the first or last item in the database whose name begins with the displayed letters. For example, if “MA” has been entered, pressing the DOWN scroll key 5n would result in the entry in the database for macadamia nuts being located and MACADAMIA NUTS being displayed on the LCD 4. If an entry is found in the database, the scale 1 moves to the display item name state 26, otherwise it remains in the display letters state 25.

Pressing the scroll buttons 5m, 5n in the display item name state 26 causes the microcontroller 11 to step forward and backwards through the database, displaying the item name for each entry visited.

If the ADD key 5l is pressed in the display item name state 26, the microcontroller 11 first determined whether the ENTER key 5o was pressed immediately before the ADD key 5l and, if not, displays a confirmation prompt on the LCD 4 requiring the user to press the ENTER key 5o to proceed. If both the ADD and ENTER keys 5l, 5o have been pressed, the specific POINTS® value for the displayed item is retrieved from the ROM 13 and multiplied by the current weight in grams derived from the current output of load cell 12. Then in a store POINTS® state 27, the resulting absolute POINTS® value is stored in the microcontroller's RAM 11e together with the identity of the item and the current measured weight, and the legend “FOOD ADDED”. Pressing of a letter key 5b, . . . , 5i in the store Points state 27 returns the scale to the display letters state 24.

Pressing the POINTS® key 5k in the display item name state 26 or the store POINTS® state 27, moves the scale 1 to a display POINTS® state 28. In the display POINTS® state 28, the microcontroller 11 retrieves the specific POINTS® value for the displayed item, if any, and multiplies it by the current measured weight in grams. To this is added POINTS® values which have been stored in the microcontroller's RAM 11e in the store POINTS® state 27 and the result is then displayed on the LCD 4 with the legend “CURRENT TOTAL”.

Pressing one of the scroll keys 5m, 5n in the display POINTS® state 28, the scale 1 moves to a show entered items state 29 in which the contents of the microcontroller's RAM 11e can be stepped through. If in this state, the POINTS® key 5k is pressed, the currently displayed entry is updated with the POINTS® value corresponding to the current measured value, in an update stored POINTS® state 30. In this way, amounts of food can be varied without having to restart the whole process from the beginning.

Following the update stored POINTS® state 30 or in response to the CANCEL key 5q being pressed in the show entered items state 29, the scale 1 returns to the display POINTS® state 28.

If the ADD key 5l is pressed in the display POINTS® state 28, the scale 1 moves to the store POINTS® state 27 in which the most recently selected item and its associated POINTS® are stored in the microcontroller's RAM 11e.

If the CLEAR key 5p is pressed in the display POINTS® state 28, the entries stored in the microcontroller's RAM 11e are deleted in a clear memory state 31. When the entries have been deleted and the scale 1 moves to the ready state 23.

If the ZERO key 5j in any of the ready, display letters and display item name states 23, 25, 26, the scale 1 enters a zeroing state 24 in which the display is zeroed and then returns to the previous state 23, 25, 26. Zeroing the display sets an offset which is subtracted from the weight corresponding to the output of the load cell 12 to provide the current weight used in the aforementioned calculations.

Thus, it can be seen that using a scale according to the present invention, a user can place a sequence of food items in the pan 3 and get a display of the accumulated POINTS® for the food items by suitable manipulation of the keys of the keypad 5.

A second embodiment of the present invention is identical to that described above with the exception of the definition of the single-valued nutritional indicator. In the second embodiment, the single-valued nutritional indicator is a value for a food item defined by:

$$p = \frac{c}{{k}_{1}} + \frac{f}{{k}_{2}} - \frac{r}{{k}_{3}}$$

where p is the resultant POINTS® value, c is the calorific value of the food item in kilocalories, f is the total fat content of the food item in grams, and r is the dietary fiber content of the food item in grams. Thus, p is monotone increasing with c and f and monotone decreasing with r. In preferred embodiments k₁>k₂>k₃. In more preferred embodiments k₁ is constant, k₂ is in the range k₁/3 to k₁/5, and k₃ is in the range k₁20 to k₁/5. For example, k₁ may be 50, k₂ may be 12, and k₃ may be 5.

In other embodiments, the nutritional parameter may be related solely to the calorific value of the food items or solely to the carbohydrate content of the food items.

While the present invention has been exemplified by numerous embodiments, the scope of the invention intended to be protected is defined by the following claims and their equivalents recognized under law.

1. A scale comprising
a weight sensor;
a database memory for storing data related to single-valued nutritional indicators for a plurality of common foodstuffs wherein the single-valued nutritional indicators are based on caloric content of the foodstuff and at least one other nutritional attribute of the foodstuff;
user input means for identifying a foodstuff being weighed;
a processor for selecting data from the database memory related to the single-valued nutritional indicator for the identified foodstuff and for calculating a single-valued nutritional indicator for the weight of the identified foodstuff as detected by the weight sensor, where the single-valued nutritional indicator is a monotone varying function per unit weight based on caloric content per unit weight and at least one other nutritional characteristic per unit weight of the foodstuff; and
means for displaying the calculated single-valued nutritional indicator for the weighed foodstuff.

2. The scale of claim 1, further comprising a writable memory and wherein the processor calculates a total single-valued nutritional indicator for plural foodstuffs placed on the weight sensor.

3. The scale of claim 2, further comprising user input means for zeroing the weight of a vessel placed on the weight sensor which vessel receives the plural foodstuffs.

4. The scale of claim 1, wherein single-valued nutritional indicators per unit weight are stored in the database and are defined by a function in which the value of the single-valued nutritional indicator per unit weight is monotone increasing with caloric content per unit weight and monotone increasing with fat content per unit weight of the foodstuff.

5. The scale of claim 4, wherein the single-valued nutritional indicator per unit weight stored in the data base memory is defined by a function in which the value p of the single-valued nutritional indicator per unit weight is given by the equation:
$$p = \frac{c}{{k}_{1}} + \frac{f}{{k}_{2}} - \frac{r}{{k}_{3}}$$
where c is the number of kilocalories per unit weight, f is the weight of fat per unit weight and r is the weight of fiber per unit weight of the foodstuff.

6. The scale of claim 5, wherein k₁>k₂>k₃.

7. The scale of claim 5, wherein
k₁, is about 50
k₂ is about 12 and
k₃ is about 5.

8. The scale of claim 1, wherein the single-valued nutritional indicator for the weighed foodstuff is displayed to an accuracy of one decimal place.

9. The scale of claim 2, wherein the total single-valued nutritional indicator for plural foodstuffs placed on the weight sensor is displayed to an accuracy of one decimal place.

10. A scale comprising
a weight sensor including means for supporting a vessel for one or more foodstuffs to be weighed;
a database for storing data employed to calculate a single-valued nutritional indicator for a plurality of common foodstuffs wherein the single-valued nutritional indicator is a monotone varying function per unit weight based on caloric content of the foodstuff and at least one other nutritional attribute of the foodstuff,
user input means for zeroing the weight of the vessel;
user input means for identifying a foodstuff being weighed;
a processor for selecting data from the database for the identified foodstuff and for calculating a single-valued nutritional indicator for the foodstuff being weighed from data selected from the database and weight data from the weight sensor; and
means for displaying the single-valued nutritional indicator.

11. The scale of claim 10, wherein the vessel is a serving plate which is sequentially loaded with plural foodstuffs and wherein the single-valued nutritional indicators of the plural foodstuffs are totaled and displayed.

12. The scale of claim 10, wherein the vessel is a dish which is sequentially loaded with plural foodstuffs comprising the ingredients of a recipe and whereas the single-valued nutritional indicators of the plural foodstuffs are totaled and displayed.

13. The scale of claim 10, wherein the single-valued nutritional indicator is calculated by multiplying a sensed weight of the foodstuff times a single-valued nutritional indicator per unit weight that is a function of the calories, fat and fiber of a unit weight of the foodstuff.

14. A method of providing a total, single-valued nutritional indicator for a food serving or recipe containing plural foodstuffs using a scale with a weight sensor, database user input, processor and display comprising:
(a) placing a food vessel on the scale;
(b) zeroing the weight reading of the weight sensor;
(c) placing a foodstuff in the vessel;
(d) entering an identification of the foodstuff using the user input;
(e) employing the processor and database to provide a selected nutritional indicator per unit weight of the identified foodstuff and calculating a single-valued nutritional indicator for the weight of the identified foodstuff sensed by the weight sensor, wherein the single-valued nutritional indicator is based on caloric content of the foodstuff and at least one other nutritional attribute of the foodstuff, where the single-valued nutritional indicator is a monotone varying function per unit weight based on caloric content per unit weight and at least one other nutritional characteristic per unit weight of the foodstuff;
(f) repeating steps (b)-(e) at least once;
(g) calculating a total single-valued nutritional indicator for the foodstuffs in the vessel;
(h) displaying the total, single-valued nutritional indicator for the foodstuffs in the vessel.

15. The method of claim 14, wherein a current total, single-valued nutritional indicator for the foodstuffs in the vessel is displayed after each foodstuff is added to the vessel.

16. The method of claim 14, wherein the single-valued nutritional indicator of a foodstuff is a function of at least the caloric and fat content of the foodstuff.

17. The method of claim 14, wherein the single-valued nutritional indicator of a foodstuff is a function of the caloric, fat and fiber content of the foodstuff.

18. The method of claim 14, wherein the user adjusts the quantity of a foodstuff to achieve a desired total, single-valued nutritional indicator of the foodstuffs in the vessel.

19. The method of claim 14, wherein the single-valued nutritional indicator per unit weight stored in the data base memory is defined by a function in which the value p of the single-valued nutritional indicator per unit weight is given by the equation:
$$p = \frac{c}{{k}_{1}} + \frac{f}{{k}_{2}} - \frac{r}{{k}_{3}}$$
where c is the number of kilocalories per unit weight, f is the weight of fat per unit weight and r is the weight of fiber per unit weight of the foodstuff.

20. The method of claim 19 wherein k₁>k₂>k₃.

21. A scale comprising
a weight sensor;
a database memory storing a database of specific nutritional parameter values for a plurality of foodstuffs;
a display;
user input means for inputing information into the scale; and
processing means configured to be responsive to operations of the user input means for selecting nutritional parameter values from the memory and generating respective absolute nutritional parameter values in dependence on an output of the weight sensor,
wherein the processing means is configured for accumulating a plurality of total nutritional parameter values for each of a plurality of foodstuffs, where the nutritional parameter value for each foodstuff is determined from a monotone increasing function per unit weight ased on caloric content per unit weight and at least one other nutritional characteristic per unit weight of the foodstuff, generated in response to a plurality of respective user input operations, using the user input means and controlling the display to display the resultant accumulated absolute nutritional parameter value, and wherein the resultant accumulated absolute nutritional parameter value is expressed as a single-valued nutritional indicator based on caloric content of the foodstuffs and at least one other nutritional attribute of the foodstuffs.

22. A scale according to claim 21, including a writable memory, wherein the processing means is configured to be responsive to a user input operation, using the user input means to store a generated absolute nutritional parameter in the writable memory, and to include absolute nutritional parameters stored in the writable memory in said accumulated nutritional value.

23. A scale according to claim 22, wherein the processing means is configured for storing a foodstuff identifier in the writable memory in association with each generated absolute nutritional parameter stored therein.

24. A scale according to claim 23, wherein the processing means is configured to be responsive to a user input operation, using the user input means, to read a foodstuff identifier and an absolute nutritional parameter value from the writable memory and control the display to display an indication of the foodstuff identified by said identifier and the associated absolute nutritional parameter value.

25. A scale according to claim 24, wherein the processing means is configured to be responsive to a user input operation, using the user input means, while said indication and associated nutritional parameter are being displayed, to update the corresponding nutritional parameter value in the writable memory in dependence on the output of the weight sensor.

26. A scale according to claim 21, wherein the processing means is configured to be responsive to a user input operation, using the user input means, to read a foodstuff identifier from database memory and control the display to display the name of the foodstuff identified by said identifier.

27. A scale according to claim 26, wherein the processing means is configured to be responsive to a user input operation, using the user input means, to read foodstuff identifiers sequentially from database memory and control the display to display in sequence the name of each foodstuff identified by one of said identifiers.

28. A scale according to claim 26, wherein the user input means includes character input means and the processing means is configured to be responsive to user in put operations, using character user means, of a sequence of characters to select read a foodstuff identifier from the database memory for which the start of the name of the foodstuff corresponds to the input character sequence.

29. A scale according to claim 21, wherein said specific nutritional parameter for a foodstuff is defined by:—
$$p = \frac{c}{{k}_{1}} + \frac{f}{{k}_{2}}$$

30. A scale according to claim 29, wherein k₂ is k₁/17.5.

31. A scale according to claim 29, wherein k₁ is about 70.

32. A scale according to claim 21, wherein said specific nutritional parameter for a foodstuff is defined by:—
$$p = \frac{c}{{k}_{1}} + \frac{f}{{k}_{2}} - \frac{r}{{k}_{\underline{3}}}$$
where c is the caloric value of the foodstuff in kilocalories per gram, f is the total fat content of the foodstuff per gram in grams, r is the dietary fiber content of the foodstuff in grams per gram, k₁ is constant, k₂ is in the range k₁/3 to k₁/5 and k₃ is in the range k₁/20 to k₁/5.

33. A scale according to claim 32, wherein k₂ is k₁/4 and k₃ is k₁/10.

34. A scale according to claim 32, wherein k₁ is about 50.
